(19) United States
(12) Patent Application Publication
(10) Pub. No.: US 2007/0208251 A1
(43) Pub. Date: Sep. 6, 2007

(54) Transformer-coupled guidewire system and method of use

(75) Inventors: Peter Traneus Anderson, Andover, MA (US); Jonathan David Schiff, Andover, MA (US)

Correspondence Address: MCANDREWS HELD & MALLOY, LTD, 500 WEST MADISON STREET, SUITE 3400, CHICAGO, IL, 60661, US

(73) Assignee: General Electric Company

(21) Appl. No.: 11/366,297

(22) Filed: Mar. 2, 2006

(51) Int. Cl. A61B 5/05; A61M 31/00; A61M 25/00

(52) U.S. Cl. 600/424; 600/585; 604/95.01

(57) ABSTRACT

Certain embodiments of the present invention provide a transformer-coupled guidewire system and method. Certain embodiments include a transmitter coil positioned in a distal end of a guidewire, a pickup coil positioned at a proximal end of the guidewire, and a first winding coiled apart from the guidewire. The guidewire is positioned with respect to the first winding such that the first winding is inductively coupled to the pickup coil to form a transformer providing power to the transmitter coil. The guidewire may include a catheter or a catheter may be positioned over the guidewire, for example. In an embodiment, the pickup coil serves as a secondary winding to form a transformer using the first winding and the pickup coil. The first winding may be coiled around a core, for example. The core may include an air gap longer than a length of the pickup coil, for example.

### RELATED APPLICATIONS

[Not Applicable]

### FEDERALLY SPONSORED RESEARCH OR DEVELOPMENT

[Not Applicable]

### MICROFICHE/COPYRIGHT REFERENCE

[Not Applicable]

### BACKGROUND OF THE INVENTION

The present invention generally relates to imaging and image-guided navigation. In particular, the present invention relates to a system and method for non-contact powering of a guidewire and/or catheter.

Medical practitioners, such as doctors, surgeons, and other medical professionals, often rely upon technology when performing a medical procedure, such as image-guided surgery or examination. A tracking system may provide positioning information for the medical instrument with respect to the patient or a reference coordinate system, for example. A medical practitioner may refer to the tracking system to ascertain the position of the medical instrument when the instrument is not within the practitioner's line of sight. A tracking system may also aid in pre-surgical planning.

The tracking or navigation system allows the medical practitioner to visualize the patient's anatomy and track the position and orientation of the instrument. The medical practitioner may use the tracking system to determine when the instrument is positioned in a desired location. The medical practitioner may locate and operate on a desired or injured area while avoiding other structures. Increased precision in locating medical instruments within a patient may provide for a less invasive medical procedure by facilitating improved control over smaller instruments having less impact on the patient. Improved control and precision with smaller, more refined instruments may also reduce risks associated with more invasive procedures such as open surgery.

Tracking systems may be ultrasound, inertial position, or electromagnetic tracking systems, for example. Electromagnetic tracking systems may employ coils as receivers and transmitters. For example, an electromagnetic tracking system may be configured in an industry-standard coil architecture (ISCA). ISCA uses three colocated orthogonal quasi-dipole transmitter coils and three colocated orthogonal quasi-dipole receiver coils. Other systems may use three large, non-dipole, non-colocated transmitter coils with three colocated orthogonal quasi-dipole receiver coils. Another tracking system architecture uses an array of six or more transmitter coils spread out in space and one or more quasi-dipole receiver coils. Alternatively, a single quasi-dipole transmitter coil may be used with an array of six or more receivers spread out in space. In an ISCA system, transmitter and receiver coil trios are precisely characterized, but individual coils may be approximately dipole, approximately collocated, and/or approximately orthogonal within the trios.

In medical and surgical imaging, such as intraoperative or perioperative imaging, images are formed of a region of a patient's body. The images are used to aid in an ongoing procedure with a surgical tool or instrument applied to the patient and tracked in relation to a reference coordinate system formed from the images. Image-guided surgery is of a special utility in surgical procedures such as brain surgery and arthroscopic procedures on the knee, wrist, shoulder or spine, as well as certain types of angiography, cardiac procedures, interventional radiology and biopsies in which x-ray images may be taken to display, correct the position of, or otherwise navigate a tool or instrument involved in the procedure.

Several areas of surgery involve very precise planning and control for placement of an elongated probe or other article in tissue or bone that is internal or difficult to view directly. In particular, for brain surgery, stereotactic frames that define an entry point, probe angle and probe depth are used to access a site in the brain, generally in conjunction with previously compiled three-dimensional diagnostic images, such as MRI, PET or CT scan images, which provide accurate tissue images. For placement of pedicle screws in the spine, where visual and fluoroscopic imaging directions may not capture an axial view to center a profile of an insertion path in bone, such systems have also been useful.

Generally, image-guided surgery systems operate with an image display which is positioned in a surgeon's field of view and which displays a few panels such as a selected MRI image and several x-ray or fluoroscopic views taken from different angles. Three-dimensional diagnostic images typically have a spatial resolution that is both rectilinear and accurate to within a very small tolerance, such as to within one millimeter or less. By contrast, fluoroscopic views may be distorted. The fluoroscopic views are shadowgraphic in that they represent the density of all tissue through which the conical x-ray beam has passed. In tool navigation systems, the display visible to the surgeon may show an image of a surgical tool, biopsy instrument, pedicle screw, probe or other device projected onto a fluoroscopic image, so that the surgeon may visualize the orientation of the surgical instrument in relation to the imaged patient anatomy. An appropriate reconstructed CT or MRI image, which may correspond to the tracked coordinates of the probe tip, may also be displayed.

A guidewire or catheter is often used in surgical procedures or other medical operations, for example. In some applications, a guidewire or catheter may be used in surgical navigation or instrument tracking. A guidewire or catheter may include a coil embedded in the tip of the guidewire or catheter, with a twisted-pair or coaxial cable running up the guidewire or catheter for use in tracking, for example. Patient safety requirements mandate that providing electricity to a twisted pair or coaxial cable be isolated from the patient. Additionally, a diameter of an electrical connection to the guidewire must be the same as a diameter of the guidewire such that a catheter may be positioned over the guidewire. Furthermore, since a sterile catheter is to be slipped over the guidewire, the entire guidewire must also remain sterile to preserve a hygienic operating environment. Thus, the connection between a power source and the guidewire must also be sterile, and whatever mates with the connection means must also be sterile.

Thus, there is a need for an improved method for supplying power to a guidewire system. There is a need for a sterile system and method for providing power to a guidewire in a hygienic environment. There is a need for a transformer-coupled guidewire system and method of use.

### BRIEF SUMMARY OF THE INVENTION

Certain embodiments of the present invention provide a transformer-coupled guidewire system and method for providing power to a guidewire coil. Certain embodiments provide a transformer-coupled guidewire system including a transmitter coil positioned in a distal end of a guidewire, a pickup coil positioned at a proximal end of the guidewire, and a first winding coiled apart from the guidewire. The guidewire is positioned with respect to the first winding such that the first winding is inductively coupled to the pickup coil to form a transformer providing power to the transmitter coil. In an embodiment, the guidewire includes a catheter. In an embodiment, a catheter is positioned over the guidewire.

Certain embodiments provide a method for non-contact powering of a guidewire coil. The method includes positioning a pickup coil on a guidewire with respect to winding apart from the guidewire, applying power to the winding, and creating a field at a second coil on the guidewire via the pickup coil.

Certain embodiments provide a transformer-coupled guidewire system. The system includes a guidewire having a coil positioned on a first end of the guidewire, and a transformer coupled to the guidewire to power the coil at the first end of the guidewire. The transformer is created from a first winding and a second winding. The first winding does not contact the guidewire. The second winding is located at a second end of the guidewire.

Certain embodiments provide a transformer-coupled medical instrument system. The system includes a medical instrument having a first coil positioned on a distal end of the instrument, and a transformer coupled to the instrument to power the first coil at the distal end of the instrument. The transformer is created from a first winding and a second winding. The first winding does not contact the instrument, and the second winding is located at a proximal end of the instrument.

### BRIEF DESCRIPTION OF SEVERAL VIEWS OF THE DRAWINGS

FIG. 1 illustrates an electromagnetic tracking system used in accordance with an embodiment of the present invention.

FIG. 2 illustrates a flow diagram for a method for tracking a position of an instrument used in accordance with an embodiment of the present invention.

FIG. 3 illustrates a guidewire system with a transformer coupling in accordance with an embodiment of the present invention.

FIG. 4 illustrates a guidewire system with a solenoidal coil in accordance with an embodiment of the present invention.

FIG. 5 illustrates a flow diagram for a method for non-contact powering of a guidewire coil used in accordance with an embodiment of the present invention.

The foregoing summary, as well as the following detailed description of certain embodiments of the present invention, will be better understood when read in conjunction with the appended drawings. For the purpose of illustrating the invention, certain embodiments are shown in the drawings. It should be understood, however, that the present invention is not limited to the arrangements and instrumentality shown in the attached drawings.

### DETAILED DESCRIPTION OF THE INVENTION

For the purpose of illustration only, the following detailed description references a certain embodiment of an electromagnetic tracking system used with an image-guided surgery system. It is understood that the present invention may be used with other imaging systems and other applications.

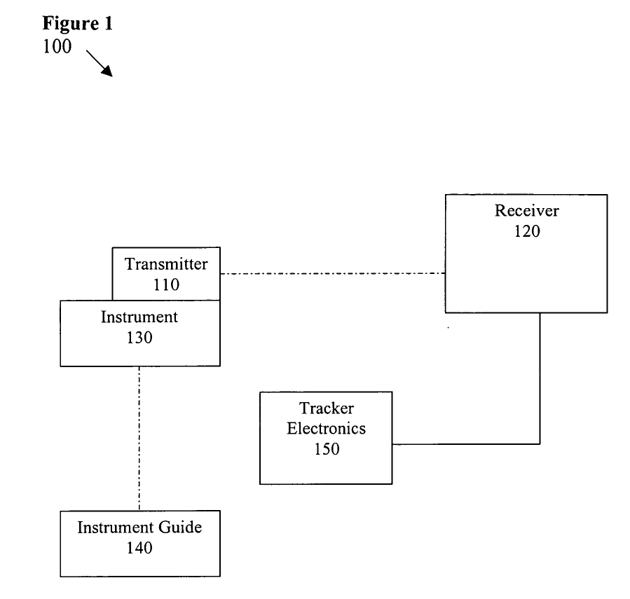

FIG. 1 illustrates an electromagnetic tracking system 100 used in accordance with an embodiment of the present invention. The tracking system 100 includes a transmitter 110, a receiver assembly 120, and a tracker electronics 150. The transmitter 110 may be a wired or wireless transmitter, for example. In an embodiment, the wireless transmitter 110 is positioned on an instrument 130. The receiver assembly 120 is located remotely from the instrument 130 and the transmitter 110. In an embodiment, an instrument guide 140 is used to control the instrument 130. The tracker electronics 150 may be integrated with the receiver assembly 120 or may be a separate module, for example. In an embodiment, the tracker electronics 150 resides on a receiver assembly 120 board to perform calculations on signal data.

In an embodiment, the receiver assembly 120 includes two receivers 122, 124. The receivers 122, 124 of the receiver assembly 120 may be receiver dipole coils or coil trios, for example. In an embodiment, the receiver assembly 120 may be a circuit board including a plurality of receiver coils. The receiver assembly 120 may be attached to the instrument guide 140. The instrument 130 may be a surgical drill or other medical instrument, for example. The instrument guide 140 may be a drill guide or other medical instrument guide, for example. In another embodiment, the instrument 130 with instrument guide 140 may be a tool that is indirectly controlled for applications wherein an operator's field of vision is obscured by an object.

In certain embodiments, the transmitter 110 is attached to the instrument 130. Alternatively, the transmitter 110 may be integrated with the instrument 130. Using the transmitter 110 and receiver assembly 120, the position of the instrument 130 is tracked with respect to the instrument guide 140 or other reference point, for example.

The system 100 may also include one or more additional transmitters (not shown) for use in instrument 130 tracking. The additional transmitter(s) may be wired or wireless transmitter(s). For example, a wireless second transmitter may be located on the instrument guide 140 or on the instrument 130. Alternatively, for example, a wired second transmitter may be located on the instrument guide 140. The second transmitter may be wired to the tracker electronics 150. A cable may be run from the instrument 130 to the tracker electronics 150. The transmitter 110 and additional transmitter(s) may be tracked simultaneously from the receivers in the receiver assembly 120.

In an embodiment, the transmitter 110 is an ISCA transmitter, such as a wireless ISCA transmitter coil trio, for example. The transmitter 110 eliminates the need for a cable connecting the instrument 130 to the tracker electronics 150. Software running with the tracker electronics 150 may be reconfigured to accommodate a wired or wireless transmitter. The transmitter 110 may draw power from the instrument 130 or may have a separate power source, for example. The transmitter 110 may be tracked from each of the receivers in the receiver assembly 120. Thus, certain embodiments use a transmitter 110 and a wired receiver assembly 120 to track the position of the instrument 130 with respect to the instrument guide 140.

In an embodiment, a gain ratio of the signal received from the transmitter 110 is known but an absolute gain in the receiver assembly 120 may not be known. The tracker electronics 150 may determine the transmitter 110 position with respect to the instrument guide 140 or other reference point. The direction or orientation of the transmitter 110 position may be determined from the received signals and gain ratio. However, a tracked position of the transmitter 110 may have range errors (i.e., the tracked position is in the right direction but not at the right distance). To determine a correct range, the tracker electronics 150 may triangulate on the tracked positions of the transmitter 110 from the receivers and use the positional relationship between the two receivers 122, 124 in the receiver array 120.

In an embodiment, the transmitter 110 is a single-coil wireless transmitter. An example of a single-coil wireless transmitter may be found at U.S. Patent Application No. 2005/0003757, entitled “Electromagnetic tracking system and method using a single-coil transmitter,” filed on Jan. 6, 2005, with inventor Peter Anderson, which is herein incorporated by reference. The wireless transmitter 110 may be a battery-powered wireless transmitter. In an embodiment, the wired receiver 120 is a twelve-coil wired receiver. Unlike a wireless receiver, the battery-powered wireless transmitter 110 does not need an auxiliary wireless channel for communicating with the receiver 120 and tracker electronics 150. A magnetic field emitted by the transmitter 110 allows both measurement of position and communication with the receiver 120 and the tracker electronics 150.

Some instruments, such as catheters, guidewires, ultrasound transducers, and flexible ear, nose and throat (ENT) instruments, may be tracked with a single small coil. In an embodiment, an instrument may be tracked with position information and without roll information.

In an embodiment, the coil of the wireless transmitter 110 is driven with a continuous wave (CW) sine wave (a 20 kHz sine wave, for example). A driver for the transmitter coil is powered by a 3 volt lithium cell, for example. The driver may be connected to the transmitter coil using a short cable (such as a 0.1 meter coaxial cable), for example. In an embodiment, the transmitter coil is 8 millimeters long and 1.7 millimeters in diameter. The transmitter coil may be wound with 7700 turns of American Wire Gauge (AWG) 54 wire around a ferromagnetic core that is 8 millimeters long and 0.5 millimeters in diameter, for example.

The core increases an effective area of the coil by a factor of approximately:

$$area\_factor = {( \frac{coil\_length}{coil\_diameter} )}^{2} . \qquad ( 1 )$$

For example, the effective coil area factor is (8 mm/1.7 mm)²=22. The coil may be a sensor coil or telecoil, such as a telecoil coil used in a hearing aid to pick up magnetic audio signals, for example.

The coil driver may not produce a precise current to drive the transmitter coil. Additionally, the effective area of the coil may not be precisely known or measured. As described below, an actual current in the coil may be calculated.

In an embodiment, the transmitter coil is small enough that the coil acts sufficiently like a dipole for tracking purposes. A dipole may be described by position, orientation, and gain (or strength). The position, orientation, and strength of the coil may be determined as described below. Therefore, the position, orientation, and gain of the wireless transmitter coil and the tracker electronics 150 may be determined without characterization.

Mutual inductance may be used in the electromagnetic tracking system to identify the positions of components in the system. Mutual inductance may allow the system to be divided into two parts: coils and electronics 150. Determining mutual inductance involves a physical design of the coils and a geometrical relationship between the coils but not details of the electronics 150 used to measure the mutual inductance. Additionally, mutual inductance does not depend on which coil receives an applied current.

In addition to the electronics 150 used to measure mutual inductance, a system including one transmitter coil and one receiver coil forms a four-terminal two-port network. A varying current injected into one coil induces a voltage in the other coil. The induced voltage V is proportional to the rate of change of the applied current I:
V=Lₘ(dI/dt) (2),
wherein Lₘ represents mutual inductance. Lₘ is based on the geometry of the coils (closed circuits). Lₘ is a ratio independent of applied current waveform or frequency. Thus, Lₘ is a well-defined property that may be measured with reasonable precision.

In an embodiment, the gain of the single transmitter coil may be determined with a plurality (e.g., 6 or more) of receiver coils. In an embodiment, a mutual inductance model provides a plurality of mutual inductances from the transmitter coil to each of the receiver coils as a function of position, orientation, and gain (POG). First, an initial estimate of POG may be selected. For example, a POG result from a previous measurement and calculation cycle may be used as an initial estimate or seed for a POG calculation. Then, an error-minimizing routine may be used to adjust the POG estimate. The POG estimate is adjusted to minimize a difference between measured and modeled mutual inductances.

A complex transmitter current (tx_current) may be expressed as, for example, a product of two factors:
tx_current=tx_current_magnitude*tx_current_phase (3),
where tx_current_magnitude is a magnitude of the transmitter 110 current, and tx_current_phase is a phase of the transmitter 110 current. In an embodiment, the magnitude of the transmitter 110 current is real, positive, and varies slowly. The magnitude of the transmitter current is proportional to the gain of the POG. Thus, transmitter current magnitude may be determined by a POG calculation. The transmitter current phase may be a complex, unity magnitude value. The phase is recalculated from newest receiver 120 signal data for each cycle. Transmitter current phase may be different for each cycle's data.

In an embodiment, a largest magnitude received signal in a receiver array is designated receiver_signal[r]. A denormalized transmitter current phase may then be calculated as follows:

$$tx\_current \_phase \_denormalized = sign \frac{receiver\_signal [ r ]}{ⅈ 2 π} , \qquad ( 4 )$$

where the sign is either +1 or −1. Then the current phase may be normalized and the sign corrected:

$$tx\_current \_phase = \frac{tx\_current \_phase \_denormalized}{\vert tx\_current \_phase \_denormalized \vert} . \qquad ( 5 )$$

A transmitter 110 complex current may then be determined:
tx_current=tx_current_mag*tx_current_phase (6).

Without a second harmonic signal measurement, a sign may be chosen for each cycle to maintain a consistent sign of the receiver_signal[n] elements over time. In an embodiment, tracking of the transmitter 110 begins from a selected position, such as a calibration position, to make an initial sign choice (+ or −). A second harmonic current of the transmitter coil may be generated with an asymmetrical waveform including even harmonics and a CW fundamental frequency. For example, a transmitter coil driver may output an asymmetrical square wave voltage (for example, ⅓, ⅔ duty cycle) to drive the coil in series with a tuning capacitor. Alternatively, a diode (or a series combination of a diode and a resistor, for example) may be connected in parallel with the coil to generate even harmonics.

A harmonic frequency may be used to determine the sign of the fundamental frequency. The harmonic may be amplitude modulated with low-speed analog or digital data without affecting a tracking function. The data may be characterization data, data from a transducer mounted on the transmitter 110, or other data, for example.

The transmitter 110 may be driven by an oscillator powered by direct current, for example. In an embodiment, the wired transmitter driver may be powered from a source of 3 volts at a milliampere direct current. For example, photocells powered by ambient light may power the driver. Alternatively, radio frequency energy may be rectified to power the driver.

In one embodiment, a single transmitter coil is located at the tip of a catheter. A small silicon photocell is connected across the coil. The photocell is illuminated with amplitude-modulated light. The photocell powers a driver for the transmitter coil. Alternatively, two photocells may be connected in antiparallel across the transmitter coil. By alternately illuminating each photocell, an alternating current may be generated in the coil.

Alternate illuminations may be achieved using two optical fibers (one to each photocell). Illumination may also be achieved using one fiber to illuminate the photocells through filters of different polarizations or different colors, for example. In another embodiment, two photocells may be integrated on top of each other. Each photocell may be sensitive to different wavelengths of light.

An optically powered coil may have advantages over an electrically powered coil. For example, optical fibers may be smaller than electrical wires. Additionally, a catheter, for example, with an optically powered coil has no electrical energy in most of the length of the catheter. An electrically powered coil may result in some electrical energy in the catheter.

In another embodiment, the receiver 120 may include an array(s) of three-axis dipole wire-wound coil trios. Due to inaccuracies in coil winding, the receiver 120 is characterized before use in tracking. The wire-wound receiver coil arrangement may have a better signal-to-noise ratio than a printed circuit board coil, due to a larger volume of copper in a wound coil of a given volume. Additionally, POG seed algorithms may be used with characterized receiver coils, for example.

In an alternative embodiment, a battery-powered wireless transmitter driver receives a clock signal from the tracker electronics 130 via a magnetic, radio frequency, ultrasonic, or other signal generator. A clock signal may eliminate phase-locking and ambiguity in the sign of the transmitter gain.

In another embodiment, the wireless transmitter 110 may be combined with various wireless radio frequency identification (RFID) schemes. RFID techniques allow for identification and/or data transfer without contact between the transmitter 110 and the receiver 120. The wireless transmitter 110 may be used with RFID technology to transmit data to the receiver 120 and tracker electronics 150.

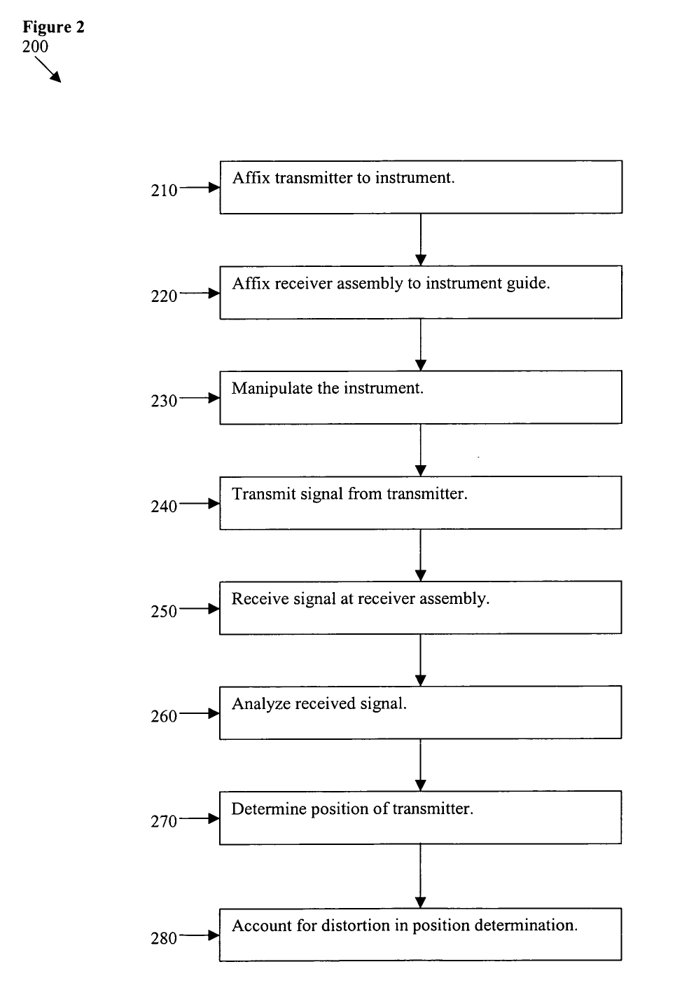

FIG. 2 illustrates a flow diagram for a method 200 for tracking a position of an instrument 130 used in accordance with an embodiment of the present invention. First, at step 210, the transmitter 110 is affixed to an instrument 130, such as a catheter, guidewire, or other medical instrument or tool. Next, at step 220, the receiver assembly 120 may be affixed to an instrument guide 140. The transmitter 110 includes one or more transmitter coils, for example. The receiver assembly 120 may include two receiver 122, 124 coils or coil trios, for example.

Then, at step 230, an operator manipulates the instrument 130 inside the patient using the instrument guide 140. At step 240, the transmitter 110 broadcasts a signal using power from the instrument 140. For example, the electronics of the transmitter 110 generate a signal using the coil of the transmitter 110.

Next, at step 250, the receivers of the receiver assembly 120 detect the signal transmitted from the transmitter 110. At step 260, the received signals are analyzed. The tracker electronics 150 measure the signals as received by the receivers 122, 124. The signals are measured based on the relationship between the receivers 122, 124 in the receiver assembly 120.

Then, at step 270, the position of the transmitter 110 is determined. The transmitter 110 position may be determined with respect to the instrument guide 140 or other reference coordinate system, for example. The direction or orientation of the transmitter 110 position may be determined from the received signals. Triangulation may determine a range to the transmitter 110 based on the tracked positions of the transmitter 110 from the receivers and on the positional relationship between the two receivers 122, 124 in the receiver array 120, for example. In an alternative embodiment, multiple transmitters transmit signals to the receiver assembly 120 to help locate the instrument 130. At step 280, distortion may be accounted for in the position determination. For example, integral (e.g., Green's function) or differential (e.g., finite-element) methods may be used to determine an impact of field effects from a distorter on the tracked position of the transmitter 110.

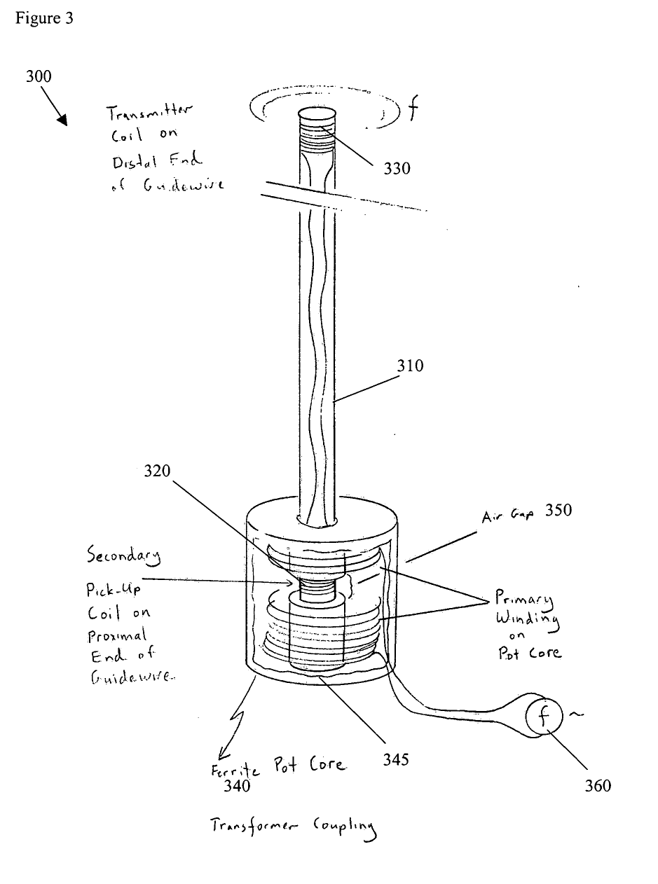

Transmitter and receiver coils, such as the coils described above, may be used in a variety of applications. FIG. 3 illustrates a guidewire system 300 with a transformer coupling in accordance with an embodiment of the present invention. A pickup coil 320 is embedded or otherwise positioned in a proximal end of a guidewire or catheter 310, for example. In an embodiment, the pickup coil 320 may be similar or identical to a transmitter coil 330 in the guidewire or catheter tip, for example. A transformer is constructed with a primary winding and magnetic core built from, for example, a pot core 340, such as a ferrite pot-core assembly. Alternatively, another cylinder or housing with a winding may be used, for example. In certain embodiments, a secondary winding, such as a removable secondary winding, in the pot core 340 may serve as the pickup coil 320 in the guidewire or catheter 310. In certain embodiments, power may be electrically provided to an outer winding in combination with the pickup coil 320 to inductively provide power to transmitter coil 330. Thus, an electrical connection to the transmitter coil 330 is replaced with a magnetic coupling, for example.

In an embodiment, a pot-core assembly 340, such as a ferrite pot-core assembly, may be used with the guidewire and/or catheter system 300. The pot-core assembly includes a bobbin, post, or pin, for example. A coil may be wound on the bobbin, post or pin, for example. The coil may be powered at a desired transmitter frequency, for example. In certain embodiments, frequency ‘f’ 360 may be configured according to tracking physics of a magnetic coupling. In certain embodiments, coils may be powered at any frequency depending upon coil size. In certain embodiments, coils are powered at a frequency ‘f’ between 25 Hz and 33 kHz, for example. In an embodiment, for example, a ferrite coil surrounds a coil on the outside of a central rod of ferrite. Alternatively, windings may be insulated copper windings or other electrically-conductive wire windings, for example. An “air-gap” 350 or region of close-to-unity magnetic permeability may be found around a central post in the pot-core. For example, flux may be added into the air in the center surrounding the guidewire 310 to form the air-gap 350. The air-gap 350 may be enlarged to be longer than the length of the pickup coil 320. A hole, such as a central axial hole 345, may be formed wide enough to pass the guidewire or catheter 310 through the hole.

In an embodiment, insulation, such as high-temperature electrical insulation, may be provided. For example, ferrite is a ferromagnetic or ferrimagnetic electrically-insulating ceramic. The air-gap may be a non-magnetic electrically-insulating ceramic, for example. High-temperature electrical insulation on magnet wire and ceramics, for example, may withstand temperatures of autoclaving to maintain a hygienic environment. Thus, the whole pot-core assembly with its moisture-sealed housing may be made autoclavable or otherwise sterilizable.

The guidewire or catheter 310 may be inserted into a hole, such as an axial hole 345, in the pot core 340 to magnetically couple power to the guidewire or catheter pickup coil 320. Power may thus be provided to a coil 330 at the tip of the guidewire or catheter 310. In an embodiment, if the guidewire or catheter 310 has two or more coils at or near its tip, a number of pot cores corresponding to the number of coils may be stacked. Each core drives a separate pickup coil to power a separate tip coil, for example. Alternatively, multiple coils may be powered using a single core. In an embodiment, separate transformer systems may be used with tracking coils on both the guidewire and the catheter to provide each of the guidewire and the catheter with a separate signal. In an embodiment, coil(s) may be wound around and/or embedded in a guidewire, catheter, and/or lead, for example. In an embodiment, coil(s) may be wound around and/or embedded in a guidewire and/or other catheter guide, and a catheter may be placed over the guidewire for insertion into a patient.

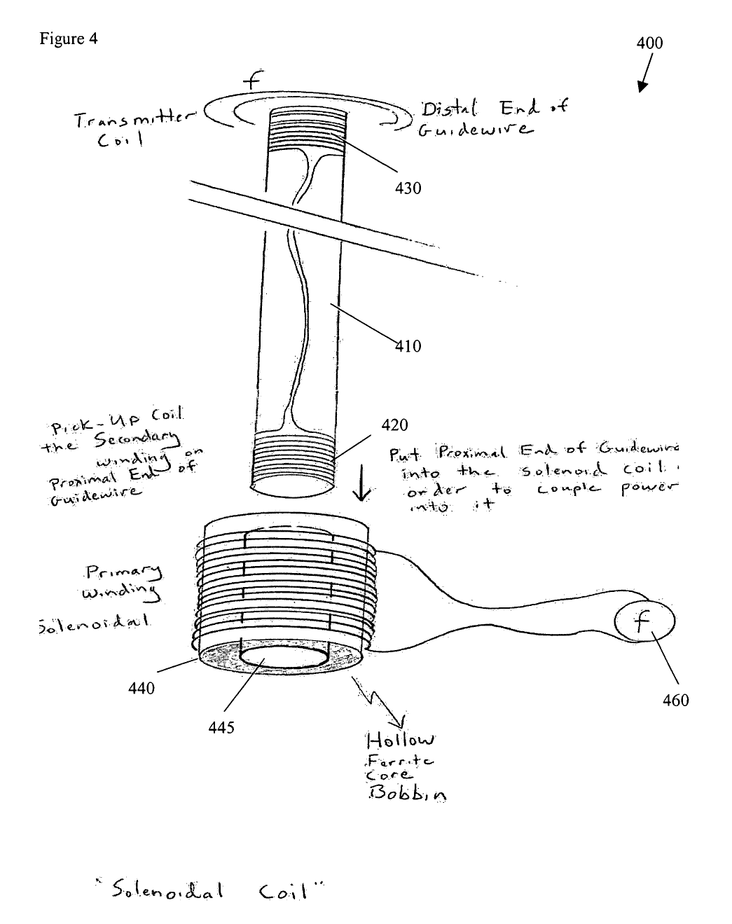

FIG. 4 illustrates a guidewire system 400 with a solenoidal coil in accordance with an embodiment of the present invention. In the guidewire system 400 of FIG. 4, the pot core is eliminated. Instead, a solenoidal coil is wound longer than the pickup coil, with a bore large enough to pass the pickup coil into the bore. In certain environments, such as in the vicinity of magnetic resonance imaging (MRI) systems, magnetic materials must be excluded. Using a solenoidal coil rather than a magnetic core may be beneficial in such cases.

The guidewire system 400 includes a transmitter coil 430 wound at a distal end of the guidewire 410. A pickup coil 420 is would at a proximal end of the guidewire 410. The proximal end of the guidewire 410 is positioned in a solenoid coil 440 in order to couple power to the guidewire 410. The solenoidal coil winding 440 serves as a primary winding. The pickup coil 420 on the guidewire 410 serves as the secondary winding. Power may be provided to the solenoidal coil 440 at frequency ‘f’ 460 in order to power the transmitter coil 430 via the pickup coil 420 on the guidewire 410.

In certain embodiments, using either the guidewire system 300 or guidewire system 400, the axial hole 445 may be open at one end. In this case, the guidewire is removed from the axial hole 445 when the catheter is slipped over the guidewire. The guidewire may be positioned by inserting the guidewire into the hole 445. Alternatively, the axial hole 445 may be open at both ends. In this case, the catheter may be slipped over the guidewire through the axial hole 445 while the guidewire remains in the axial hole 445. Thus, the catheter may be slid over the guidewire without taking the catheter out of the transformer. In an embodiment, the guidewire is fed through the transformer and into a patient. The catheter is slid or otherwise positioned over the guidewire into the patient. The guidewire may be approximately centered in the transformer to allow tracking of the guidewire and/or catheter while manipulating the catheter in the patient.

Thus, power may be provided to the guidewire or catheter or without electrical contact or wires between coils. Power may be provided to the guidewire or catheter without creating bulges in the guidewire or catheter and without disturbing the hygienic surgical environment. The guidewire or catheter may be autoclaved or otherwise sterilized without affecting the power-generating coils, for example. In certain embodiments, a transformer assembly may provide power to the guidewire in a manner that does not increase the diameter of the guidewire. The transformer assembly is sterilizable to preserve the sterility of the entire length of the guidewire, for example.

In an embodiment, imparting a magnetic flux in one coil generates a current flow between coils which produces flux at the other coil. A transformer is formed with the coil in the proximal end of the guidewire serving as the secondary coil of the transformer. A primary winding assembly is positioned with respect to the proximal end of the guidewire to couple the guidewire coil to the magnetic flux from the primary winding assembly. In an embodiment, a guidewire may be positioned in and out of the pot core or other winding, and a catheter may slide over the guidewire. Certain embodiments, allow the guidewire and/or catheter to be tracked and provide power to the guidewire/catheter, for example.

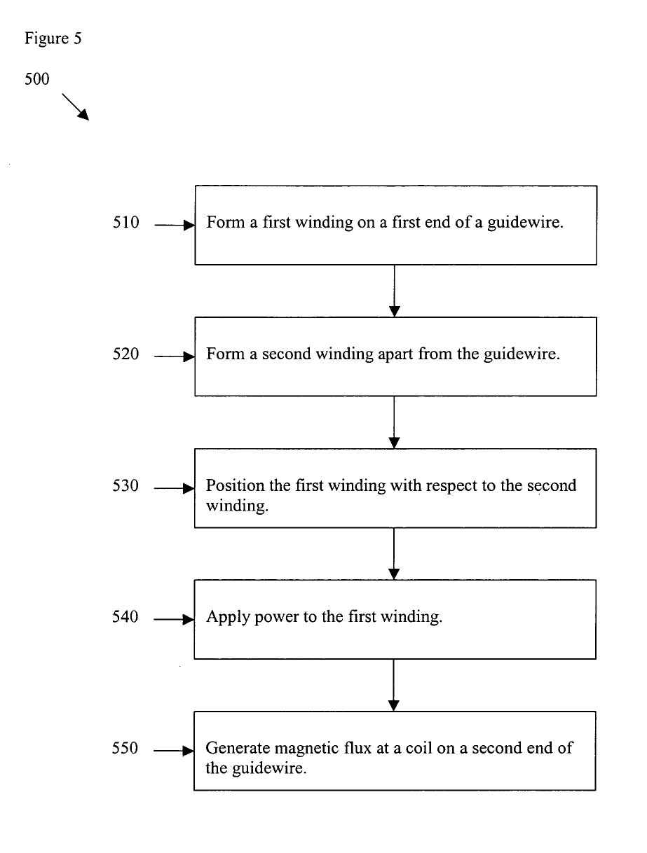

FIG. 5 illustrates a flow diagram for a method 500 for non-contact powering of a guidewire coil used in accordance with an embodiment of the present invention. At step 510, a first winding is formed on a first end of a guidewire. For example, a pickup coil may be wound at a proximal end of a guidewire and/or guidewire/catheter combination. At step 520, a second winding is formed apart from the guidewire, such as around a core.

At step 530, the first winding on the guidewire is positioned with respect to the second winding. For example, the proximal end of the guidewire is positioned inside the core such that the primary and secondary windings form a transformer to provide power to the guidewire. At step 540, power is applied to the first winding. For example, the first winding is powered at a frequency (f) via the second winding. At step 550, corresponding magnetic flux is generated at a coil on a second end of the guidewire. For example, a transmitter coil at a distal end of the guidewire may be powered due to the power applied to the pickup coil at the proximal end of the guidewire. The transmitter coil may be used to track the guidewire and/or catheter, for example. Alternatively or in addition, power may be generated without contact on the guidewire to operate an ultrasound transducer or other instrument, for example.

In another embodiment, an instrument, such as a surgical drill bit or other device, may be used instead of and/or in addition to a guidewire described above. Coils may be positioned on both ends of the instrument, similar to the systems and methods described above in relation to a guidewire/catheter system. Using a surgical drill bit, for example, a distal end of the bit may be used to cut into bone in a patient by rotating the bit using, for example, a motor. The coil in the distal end tracks the location of the cutting, even if the bit should flex. The coil in the proximal end is powered magnetically as described above for the guidewire. For the bit, the magnetic coupling helps ensure that the rotation of the bit is not hindered by a non-rotating pot core or solenoid, for example.

Thus, certain embodiments provide an ability to couple power into a guidewire and/or instrument without increasing its diameter or requiring any kind of connector. Alternatively, electrical contacting, optical coupling, or a passive transponder may be used to provide power to a guidewire and/or instrument. Certain embodiments provide a non-contacting method for powering a coil used for tracking and/or other purposes in surgical navigation, for example. Certain embodiments form a transformer to couple electrical energy to a guidewire coil isolatedly for use in tracking, power, and/or other application, for example. Power may be applied to one or more tracking coils in a guidewire and/or catheter or instrument, for example to facilitate tracking. Alternatively and/or in addition, power may be applied to a guidewire and/or catheter to power an instrument, such as an ultrasound transducer, in contact with the guidewire and/or catheter.

While the invention has been described with reference to certain embodiments, it will be understood by those skilled in the art that various changes may be made and equivalents may be substituted without departing from the scope of the invention. In addition, many modifications may be made to adapt a particular situation or material to the teachings of the invention without departing from its scope. Therefore, it is intended that the invention not be limited to the particular embodiment disclosed, but that the invention will include all embodiments falling within the scope of the appended claims.

1. A transformer-coupled guidewire system, said system comprising:
a transmitter coil positioned in a distal end of a guidewire;
a pickup coil positioned at a proximal end of said guidewire; and
a first winding coiled apart from said guidewire,
wherein said guidewire is positioned with respect to said first winding such that said first winding is inductively coupled to said pickup coil to form a transformer providing power to said transmitter coil.

2. The system of claim 1, wherein said guidewire is incorporated with a catheter.

3. The system of claim 1, wherein a catheter is positioned over said guidewire.

4. The system of claim 1, where said transmitter and said pickup coil are embedded in said guidewire.

5. The system of claim 1 further comprising a plurality of transmitter coils and pickup coils in said guidewire.

6. The system of claim 1, wherein said pickup coil serves as a secondary winding to form a transformer using said first winding and said pickup coil.

7. The system of claim 1, wherein said first winding is coiled around a core.

8. The system of claim 7, wherein said guidewire is positioned in an axial hole in said core.

9. The system of claim 8, wherein said axial hole is open at least one end.

10. The system of claim 7, wherein said core comprises a pot core.

11. The system of claim 7, wherein said core includes an air gap longer than a length of said pickup coil.

12. The system of claim 1, wherein said first winding comprises a solenoidal coil.

13. A method for non-contact powering of a guidewire coil, said method comprising:
positioning a pickup coil on a guidewire with respect to a winding apart from said guidewire;
applying power to said winding; and
creating a field at a second coil on said guidewire via said pickup coil.

14. The method of claim 13, wherein said positioning step further comprises positioning said guidewire in an axial hole of a core including said winding such that said pickup coil and said winding act as a transformer to power said coil.

15. The method of claim 13, further comprising tracking said guidewire using said second coil.

16. The method of claim 13, further comprising powering an instrument attached to said guidewire using said second coil.

17. The method of claim 13, further comprising positioning a catheter over said guidewire.

18. A transformer-coupled guidewire system, said system comprising:
a guidewire having a coil positioned on a first end of said guidewire; and
a transformer coupled to said guidewire to power said coil at said first end of said guidewire, wherein said transformer is created from a first winding and a second winding, wherein said first winding does not contact said guidewire and wherein said second winding is located at a second end of said guidewire.

19. The system of claim 18, wherein said transformer-coupled guidewire system is shielded to withstand sterilization.

20. The system of claim 18, wherein said guidewire is isolated from contact with a power source to preserve a hygienic environment.

21. A transformer-coupled medical instrument system, said system comprising:
a medical instrument having a first coil positioned on a distal end of said instrument; and
a transformer coupled to said instrument to power said first coil at said distal end of said instrument, wherein said transformer is created from a first winding and a second winding, wherein said first winding does not contact said instrument and wherein said second winding is located at a proximal end of said instrument.

22. The system of claim 21, wherein said instrument comprises a surgical drill bit.
